(19) United States
(12) Patent Application Publication
(10) Pub. No.: US 2004/0197333 A1
(43) Pub. Date: Oct. 7, 2004

(54) Use of TGF-beta antagonists to inhibit tumor cell formation or progression

(75) Inventors: Manikkam Suthanthiran, Scarsdale, NY (US); Minoru Hojo, New York, NY (US)

Correspondence Address: HOFFMANN & BARON, LLP, 6900 JERICHO TURNPIKE, SYOSSET, NY, 11791, US

(73) Assignee: Cornell Research Foundation, Inc.

(21) Appl. No.: 10/830,647

(22) Filed: Apr. 23, 2004

Related U.S. Application Data
(62) Division of application No. 09/780,953, filed on Feb. 9, 2001, now abandoned.
(60) Provisional application No. 60/181,526, filed on Feb. 10, 2000.

(51) Int. Cl. A61K 39/395

(52) U.S. Cl. 424/145.1

(57) ABSTRACT

The invention provides a method for reducing formation or progression of neoplasms associated with immunosuppressive therapy in a mammal, the method comprising treating the mammal with an effective amount of a TGF-β antagonist. Also provided are compositions comprising a TGF-β antagonist and an immunosuppressive agent such as cyclosporin or FK506. The TGF-β antagonists of the invention include proteins and polypeptides that bind TGF-β receptors, anti-TGF-β antibodies, TGF-β receptors and fragments thereof. In another aspect the invention provides methods for identifying compounds capable of inhibiting the formation or proliferation of tumors in a mammal undergoing immunosuppressive therapy.

This work was supported at least in part by a grant award from the National Institutes of Health. The government may have certain rights to this invention.

### BACKGROUND

Malignancy is a common and dreaded complication following organ transplantation (References 1-4). The high incidence of neoplasm and its aggressive progression, which are associated with immunosuppressive therapy, were previously believed to be due to the inability of the suppressed immune system to eliminate cancerous cells (neoplasms). See for example, Novak et al., Nature Medicine 5:382 (1999).

Immunosuppressive therapy is currently a practical necessity in all organ transplantation recipients except those in which the donor and recipient are identical twins. Drugs typically employed as immunosuppressive agents include azathioprine, steroids, cyclosporine, antilymphocyte globulins, and monoclonal anti-T cell antibodies.

Cyclosporine, a cyclic peptide (also known as cyclosporin A or CsA) and the macrolide FK506 (tacrolimus) are well known and widely used immunosuppressants for transplant recipients and autoimmune patients. Cyclosporine has had major impact on improving patient outcome following organ transplantation (References 4 and 5). Other drugs typically employed as immunosuppressive agents include cyclosporine, azathioprine, leflunomide, rapamycin and other immunophilin (FKBP) targeted compounds including dexamethasone; also included in this group are steroids including corticosteroids, antilymphocyte globulins, and monoclonal anti-T cell antibodies.

Transforming growth factor β (TGF-β) belongs to a family of multifunctional cytokines which regulate normal cell growth, development, and tissue remodeling following injury. TGF-β has been reported to play a major role in the pathogenesis of fibrotic disease such as renal, cardiac and vascular end organ damage associated with hypertension and diabetes.

TGF-β has also been identified as playing a complex role in human cancer. Its expression is elevated in a variety of cancer types and it has been reported to exert its effects through a variety of mechanisms, both paracrine and autocrine. (Ananth et al., Cancer Research 59:2210-16 (1999); Arteaga et al., J. Natl. Cancer Inst. 91:46-53 (1999); Wojtowicz-Praga et al., J. Immunotherapy, 19:169-175 (1996); Ohmori et al., Exp. Cell Res. 245:350-359 (1998); and Won et al., Cancer Res., 59:1273-1277 (1999)).

From the foregoing it is clear that there is a need to protect patients undergoing immunosuppressive therapy from increased risk of acquiring or of exacerbating the progression of preexisting malignant conditions.

### SUMMARY OF THE INVENTION

In one embodiment the invention provides methods for reducing formation or progression of neoplasms associated with immunosuppressive therapy in a mammal. The methods comprise treating the mammal with an effective amount of a TGF-β antagonist.

Also provided are compositions for use in these methods. The compositions comprise an effective amount of a TGF-β antagonist and an immunosuppressive agent.

In addition, the invention provides methods for identifying compounds capable of inhibiting the formation or proliferation of tumors in a mammal undergoing immunosuppressive therapy. These methods comprise: first providing a test animal with a tumor cell; second, treating the test animal with an immunosuppressive agent in an immunosuppressive regimen, followed by administering the TGF-β antagonist candidate test compound to the test animal; third, monitoring the growth of the tumor cell in the test animal; and then comparing the growth of the tumor cell in the test animal with the growth of a tumor cell inoculated into a control animal undergoing an identical immunosuppressive regimen, but which has not been treated with the test compound.

### BRIEF DESCRIPTION OF THE FIGURES

FIG. 1 shows the TGF-β concentrations (mean±s.d.) in supematants obtained from untreated or cyclosporine-treated A-549 cells. The cells were incubated for 72 h, in the absence or presence of 0.5 or 0.1 μgml⁻¹ cylosporine, and a sandwich ELISA assay (ref. 7) was used to quantify TGF-β levels.

FIG. 2 shows the effects of CsA treatment on the motility of A-549 cells.

FIG. 3 shows the changes in proliferation of A-549 cells after cyclosporine treatment. Panel (a) shows cyclosporine-associated inhibition of anchorage-dependent proliferation. Panel (b) shows cyclosporine-induced stimulation of anchorage-independent growth.

FIG. 4 shows potential mechanisms for cyclosporine-associated tumor progression. In this formulation, cyclosporine induced TGF-β production by tumor cells promotes cell invasiveness by a cell-autonomous mechanism that is independent of and/or complementary to cyclosporine's immunosuppressive effect on the host's immune system.

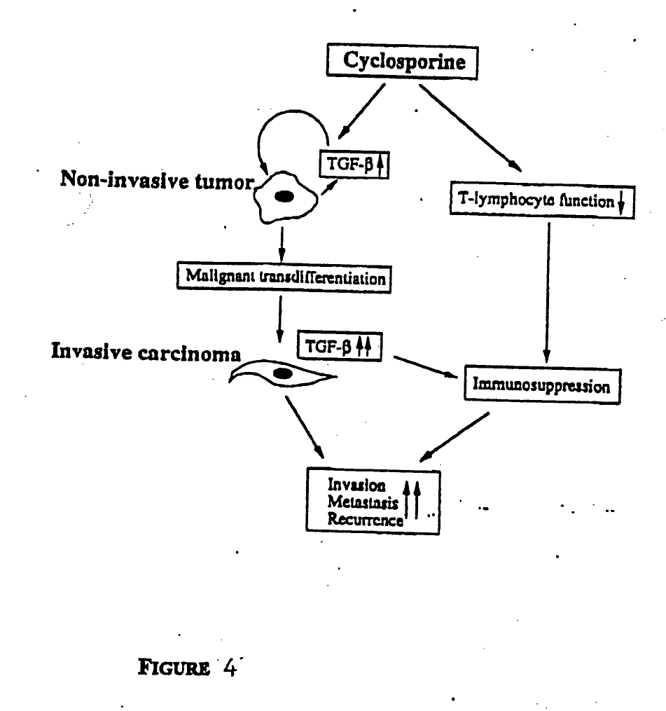

### DETAILED DESCRIPTION OF THE INVENTION

The present invention is based on the novel finding that cyclosporine A, in addition to being an immunosuppressant that may prevent treated patients from mounting an adequate immune response to cancer cells, also causes morphological changes in cancer cells that are associated with a more invasive phenotype. These changes include membrane ruffling, formation of pseudopodia and an increased ability to metastasize in vivo.

Surprisingly, the present inventors have discovered that these direct effects are mediated by TGF-β, and that antagonists of TGF-β are able to prevent cyclosporine-induced increases in metastases. Moreover, the anti-tumor effects of TGF-62 antagonists are also evident in SCID-beige mice, which are deficient in T cells, B cells and natural killer cells. Thus, the ability of TGF-β antagonists to block cyclosporine-induced phenotypic changes in cancer cells is independent of the host immune system.

Based on this discovery, the invention provides methods for reducing formation or progression of a neoplasm associated with immunosuppressive therapy in a mammal by treating with a TGF-β antagonist. “Reducing formation or progression of a neoplasm” as used herein means prevention or inhibition of initiation, establishment, proliferation or metastasis of the neoplasm.

Neoplasms include all forms of cancer cells and cell masses known as tumors, which may be malignant or benign, and may be invasive or non-invasive. Tumors include solid tumors and disseminated tumors. For example, disseminated tumors include lymphomas and leukemias and the like. Other tumors, such as solid tumors, include without limitation, adenocarcinomas, carcinomas, myelomas, melanomas, gliomas, sarcomas, adenosarcomas, adenomas and the like.

Tumors can occur in virtually all parts of the human body, including every organ. The tumors may, for example, be present in the breast, heart, lung, small intestine, colon, spleen, kidney, bladder, head and neck, ovary, prostate, brain, pancreas, skin, bone, bone marrow, blood, thymus, uterus, testicles, cervix, and liver. These examples are included for illustrative purposes and are not intended to be limiting in any way.

The neoplasms treatable by the methods of the present invention are associated with immunosuppressive therapy and may occur at any stage of the therapy. The term “associated with the immunosuppressive therapy” means these neoplasms may be potentiated, formed, progress, proliferate or metastasise during or subsequent to the immunosuppressive therapy. For example, the neoplasms may be caused or mediated by the immunosuppressive therapy.

The methods of the present invention comprise treating the mammal with an effective amount of a TGF-β antagonist. As used herein the term “TGF-β ” may be any one or more of the TGF-β isoforms, including TGF-β1, TGF-β2 and TGF-β3.

The TGF-β antagonist may be administered at any time relative to the immunosuppressive therapy. For example, the methods of the invention include pretreatment with a TGF-β antagonist; contemporaneous treatment with a TGF-β antagonist and immunosuppressive therapy, or treatment with a TGF-β antagonist after immunosuppressive therapy, or any combination resulting in overlapping treatment.

The immunosuppressant used for therapy is not a limiting feature of the invention and may be any immunosuppressant which is capable of altering the expression or regulation of TGF-β. Immunosuppressants are currently used in treatment of transplant recipients and autoimmune patients (e.g. those suffering from rheumatoid arthritis, lupus erythamatosis, Crohn's disease and inflammatory bowel disease). Such immunosuppresants include cyclosporine; FK506 (for a review, see for example Adler et al. Curr Opin Nephrol Hypertens 7(5):539-45 (1998)); azathioprine (reviewed in Gaffney & Scott Br J Rheumatol 37(8):824-36 (1998)); Leflunomide (see Prakash & Jarvis Drugs 58(6): 1137-64); Rapamycin and analogs (see for example U.S. Pat. Nos. 5,985,890; 5,998,408; 6,015,815 and 6,022,890) or any compound which mimics FK506 in binding to immunophilin (FKBP: see U.S. Pat. No. 5,968,802); steroids, particularly corticosteroids and dexamethasone or any other compounds which stimulate T-cells to produce TGF-β; anti-lymphocyte globulins and monoclonal anti-T cell antibodies, or any other immunosuppressant that induces TGF-β.

As used herein, a “TGF-β antagonist” is any molecule which is able to decrease the amount or activity of TGF-β, either within a cell or within a physiological system. The ability to decrease the amount or activity of TGF-β is not limited by any mechanism. For example, a TGF-β antagonist may be a molecule which inhibits expression of TGF-β at the level of transcription, translation, processing, or transport; it may affect the stability of TGF-β or conversion of the precursor molecule to the active, mature form; it may affect the ability of TGF-β to bind to one or more of its receptors (Type I, Type II or Type III); or it may interfere with TGF-β signaling.

A variety of TGF-β antagonists and methods for their production are well known in the art and many more are currently under development. The specific TGF-β antagonist employed is not a limiting feature, as any effective TGF-β antagonist may be useful in the methods of this invention. Examples of such antagonists include monoclonal and polyclonal antibodies directed against one or more isoforms of TGF-β (U.S. Pat. No. 5,571,714 and PCT patent application WO 97/13844), TGF-β receptors, fragments thereof, derivatives thereof and antibodies directed against TGF-β receptors (U.S. Pat. Nos. 5,693,607, 6,008,011, 6,001,969 and 6,010,872 and PCT patent applications WO 92/00330, WO 93/09228, WO 95/10610 and WO 98/48024); latency associated peptide (WO 91/08291), large latent TGF-β (WO 94/09812), fetuin (U.S. Pat. No. 5,821,227), decorin and other proteoglycans such as biglycan, fibromodulin, lumican and endoglin (U.S. Pat. Nos. 5,583,103, 5,654,270, 5,705,609, 5,726,149, 5,824,655 5,830,847, 6,015,693 and PCT patent applications WO 91/04748, WO 91/10727, WO 93/09800 and WO 94/10187).

Further examples of such antagonists include somatostatin (PCT patent application WO 98/08529), mannose-6-phosphate or mannose-1-phosphate (U.S. Pat. Nos. 5,520,926), prolactin (PCT patent application WO 97/40848), insulin-like growth factor II (PCT patent application WO 98/17304), IP-10 (PCT patent application WO97/00691), arg-gly-asp containing peptides (U.S. Pat. No. 5,958,411 and PCT patent application WO 93/10808 and), extracts of plants, fungi and bacteria (European patent application 813875, Japanese patent application 8119984 and U.S. Pat. No. 5,693,610), antisense oligonucleotides (U.S. Pat. No. 5,683,988, 5,772,995, 5,821,234 and 5,869,462 and PCT patent application WO 94/25588), and a host of other proteins involved in TGF-β signaling, including SMADs and MADs (European patent application EP 874046, PCT patent applications WO 97/31020, WO 97/38729, WO 98/03663, WO 98/07735, WO 98/07849, WO 98/45467, WO 98/53068, WO 98/55512, WO 98/56913, WO 98/53830, and WO 99/50296, and U.S. Pat. Nos. 5,834,248, 5,807,708 and 5,948,639) and Ski and Sno (G. Vogel, Science, 286:665 (1999) and Stroschein et al., Science, 286:771-74 (1999)) and fragments and derivatives of any of the above molecules that retain the ability to inhibit the activity of TGF-β.

TGF-β receptors and TGF-β-binding fragments of TGF-β receptors, especially soluble fragments are useful TGF-β antagonists in the methods of the present invention. TGF-β receptors and the nucleic acids encoding them are well known in the art. The nucleic acid sequence encoding TGF-β type 1 receptor is disclosed in GENBank accession number L15436 and in U.S. Pat. No. 5,538,892 of Donahoe et al. The nucleic acid sequence of TGF-β type 2 receptor is publicly available under GENBank accession numbers AW236001; AI35790; AI279872; AI074706; and AA808255. The nucleic acid sequence of TGF-β type 3 receptor is also publicly available under GENBank accession numbers NM 003243; AI887852; AI817295; and AI681599.

In a preferred embodiment, the TGF-β antagonist is an antibody that blocks TGF-β binding to its receptor, or fragments thereof such as F(ab)₂ fragments, Fv fragments, single chain antibodies and other forms of “antibodies” that retain the ability to bind to TGF-β. The antibody may be chimerized or humanized. In this specification, a chimerized antibody comprises the constant region of a human antibody and the variable region of a non-human antibody, such as a murine antibody. A humanized antibody comprises the constant region and framework variable region (i.e. variable region other than the hypervariable region) of a human antibody and the hypervariable region of a non-human antibody, such as a murine antibody. Of course, the antibody can be any other type of antibody derivative, such as a human antibody selected or screened from a phage display system or produced from a xenomouse.

In a more preferred embodiment, the monoclonal antibody is a humanized form of the murine monoclonal antibody 1D11.

In a related embodiment, the TGF-β antagonist is delivered by means of gene therapy, wherein a nucleic acid sequence encoding the antagonist is administered to the patient in vivo or to cells in vitro which are then introduced into a patient, and the antagonist is produced by expression of the produce encoded by the nucleic acid sequence. Methods for gene therapy to deliver TGF-β antagonists are also well known to those of skill in the art. See, for example, PCT patent application WO 96/25178.

As used herein, an “effective amount” of a TGF-β antagonist is an amount effective to detectably reduce the establishment, formation, progression or metastasis of neoplasms in vivo in a mammal. For example the establishment, formation, progression or metastasis of neoplasms in vivo in a mammal may be reduced by at least 10%, preferably the reduction may be at least about 25%, more preferably the reduction may be at least about 50% and still more preferably the reduction may be at least about 75%. Yet more preferably the reduction may be at least about 90% and more preferably still the reduction may be at least about 99%. Optimally, the establishment, formation, progression or metastasis of neoplasms in vivo in a mammal may be reduced by 100%, i.e. completely inhibited. For instance the complete inhibition may be achieved in the methods of the present invention by using a neutralizing antagonist.

TGF-β activity may be readily measured in a variety of in vitro and in vivo assays. For example, in vitro assays include those based on the ability of TGF-β to effect morphological changes or induce anchorage-independent growth of non-transformed cells (Roberts et al., P.N.A.S., 78:5339-5343 (1981)), the IL-1 and PHA-dependent thymocyte assay of Ellingsworth et al. (Cell Immunol., 114:41 (1988)), the ability of TGF-β to inhibit the proliferation of mink lung cells (D. Danielpour et al. J. Cellular Physiol. 138:79-86 (1989)), and any other assay which is useful to measure TGF-β activity. Examples of in vivo assays are set forth herein. By using the teachings set forth herein, the skilled artisan may, without undue experimentation, correlate the degree of inhibition of TGF-β activity in in vitro assays with its ability to produce the desired physiological effect in vivo, e.g., in animal models of neoplasms associated with immunosuppressive therapy. For example, the present inventors measured the ability of a TGF-β antagonist to reduce the morphologic changes in cancer cells that are associated with exposure to immunosuppresive agents. This reduction in morphologic changes correlated with a reduced ability to metastasize in vivo.

Other parameters of TGF-β activity may also be used in order to determine effective amounts of a TGF-β antagonist to be used in the methods of this invention. Such amounts may vary depending on the specific TGF-β antagonist selected, the time of administration (either before, during or after immunosuppressive therapy), a variety of factors and conditions related to the subject to be treated such as age, weight, health, etc., and, if the neoplasm has already occurred, the type and severity of the neoplasm.

Dose response curves and toxicity data obtained in preclinical animal work would also be among the factors routinely considered in determining the optimum dose for a given patient. Given the teachings set forth herein, the determination of an effective amount of a TGF-β antagonist is routine and well within the ability of those ordinarily skilled in the art.

The method of the present invention is applicable to any mammal. Examples of mammals to which the method may be usefully applied include laboratory animals, including rodents such as mice, rats and guinea pigs; farm animals such as cows, sheep, pigs and goats; pet animals such as dogs and cats; and primates such as monkeys, apes and humans. The invention is most preferably applied in human clinical situations, particularly where the patient is undergoing immunosuppressive therapy after organ or tissue transplantation, or any other form of surgery where suppression of the immune system of the patient is indicated. However, other mammals may also benefit from the practice of the invention. These other high value animals such as horses and fur animals such as mink.

The therapeutic methods of the present invention may be combined with traditional cancer treatments. Treatments of cancer traditionally include chemotherapy or radiation therapy. Some examples of chemotherapeutic agents include doxorubicin, cisplatin, and taxol. The radiation can be either from an external beam or from a source placed inside a patient, i.e., brachytherapy.

In another embodiment, the invention provides compositions comprising a pharmaceutically effective amount of a TGFβ antagonist and an immunosuppressive agent. Such compositions are preferably formulated in a pharmaceutically acceptable carrier and may be delivered by any method, including without limitation, orally, topically or parenterally (e.g. by intravenous, intradermal, subcutaneous and transmucosal routes etc.) alone or in conjunction with other pharmaceuticals. Any of the TGF-β antagonists identified above is useful the compositions of this invention.

In another embodiment the present invention provides a method of identifying compounds capable of inhibiting the formation or proliferation of tumors in a mammal undergoing immunosuppressive therapy. The method comprises: first providing a test animal with a tumor cell; second treating the test animal with an immunosuppressive agent in an immunosuppressive regimen; third administering a TGF-β antagonist candidate to the test animal; then monitoring the growth of the tumor cell in the test animal; and finally, comparing the growth of the tumor cell in the test animal with the growth of the tumor cell inoculated into a control animal.

The test animals may be any mammal, particularly laboratory animals such as rats or mice, and preferably the animals are immune deficient animals. SCID/SCD and SCID-beige mice are particularly well suited for this method. The tumor cells may be any tumor cells, particularly murine cells e.g. renca cells (renal adenocarcinoma cells); Lewis Lung carcinoma cells; mammary gland epithelial cells (e.g. NmuMG cells); mink lung epithelial cells (e.g. CCL-64 cells). Human tumor cells are particularly preferred, including for instance, human bladder transitional carcinoma cell (HTB, human lung adenocarcinoma cells such as A-549 cells, and human bladder cancer cell lines such as T24 cells).

In a particularly advantageous embodiment the screening method of the present invention the test animal is a SCID-beige mouse and the tumor cells is a tumor cell selected from the following: renca cells (renal adenocarcinoma cells); Lewis Lung carcinoma cells; mammary gland epithelial cells (e.g. NmuMG cells); mink lung epithelial cells (e.g. CCL-64 cells). Human tumor cells are particularly preferred, especially one of the following: human bladder transitional carcinoma cells (HTB, human lung adenocarcinoma cells such as A-549 cells, and human bladder cancer cell lines such as T24 cells). In the preferred embodiment the growth of the tumor cells that is monitored is anchorage-independent growth.

The disclosures of all of the patents and other documents cited above reflect the state of the art and may be useful in practicing the full scope of the present invention. All are hereby incorporated by reference.

General Methods

Antibody preparation. Antibodies include polyclonal, monoclonal and recombinant antibodies, including single chain antibodies and antigen-binding fragments of each. Antibodies may be prepared by a wide variety of techniques ranging from techniques including in vivo inoculation, isolation of hybridomas, xenograft techniques, selection or screening based on phage display and many others well known to those of skill in the art. For example, fragments such as Fab fragments may be prepared in accordance with the method of Huse et al., Science 246, 1275-1281 (1989) and Coligan, J. E. et al. (Eds.) Current Protocols in Immunology, Wiley Intersciences, New York, (1999).

Many methods for preparartion of antibodies are routinely available, the following description is merely exemplary and not intended to be limiting in any way. The antibodies are preferably monoclonal. Monoclonal antibodies may be produced by methods known in the art. These methods include the immunological method described by Kohler and Milstein in Nature 256:495-497 (1975) and by Campbell in “Monoclonal Antibody Technology, The Production and Characterization of Rodent and Human Hybridomas” in Burdon et al. (Eds.), Laboratory Techniques in Biochemistry and Molecular Biology, Volume 13, Elsevier Science Publishers, Amsterdam (1985); and Coligan, J. E, et al. (Eds.), Current Protocols.in hmmunology, Wiley Intersciences, New York, (1999); as well as the recombinant DNA method described by Huse et al., Science 246:1275-1281 (1989).

In order to produce monoclonal antibodies, a host mammal is inoculated with a peptide or peptide fragment as described above, and then boosted. Spleens are collected from inoculated mammals a few days after the final boost. Cell suspensions from the spleens are fused with a tumor cell in accordance with the general method described by Kohler and Milstein in Nature 256:495-497 (1975). See also Campbell, “Monoclonal Antibody Technology, The Production and Characterization of Rodent and Human Hybridomas” in Burdon et al. (Eds.), Laboratory Techniques in Biochemistry and Molecular Biology, Volume 13, Elsevier Science Publishers, Amsterdam (1985) and Coligan, J. E., et al. (Eds.), Current Protocols in Immunology, Wiley Intersciences, New York, (1999)). These examples are provided for illustration purposes and should not be construed as limiting in any way. Many other methods are equally applicable and those of skill in the art will recognize that a wide variety of other methods that may be used in the present invention.

Preparation of Peptides, Polypeptides and Proteins. Peptides, polypeptides and proteins may be prepared by many different techniques well known to all those of skill in the art. For example, the peptides, polypeptides and even some proteins may be chemically synthesized. Alternatively, the peptides, polypeptides or proteins may be prepared as recombinant molecules or purified from natural sources by methods well known in the art. For example, recombinant form of a protein such as a receptor protein may be prepared by providing DNA that encodes the protein; amplifying or cloning the DNA in a suitable host; expressing the DNA in a suitable host; and harvesting the protein. See inter alia Sambrook, J. et al. (eds), Molecular Cloning, Second Edition, Cold Spring Harbor Laboratory Press, Cold Spring Harbor, N.Y. (1989) and Ausubel, F. M. et al. (eds), Current Protocols in Molecular Biology, John Wiley & Sons, New York (1999). Again these examples are for illustration and are not to be taken as limiting the scope of the invention.

### EXAMPLES

Cell line and culture. Human lung adenocarcinoma cells, A-549 cells (ATCC CCL 185, American Type Culture Collection, Rockville, Md.), human bladder transitional cell carcinoma cells (ATCC HTB4, T24), mink lung epithelial cells, CCL-64 (ATCC), mouse mammary gland epithelial cells, NMuMG (ATCC), Lewis lung carcinoma cells (ATCC), were grown in minimum essential medium (MEM) supplemented with 10% fetal bovine serum, at 37° C. in a 95% air-5% CO₂ atmosphere. Murine renal adenocarcinoma cells, Renca cells (a gift from Dr. R. H. Wiltrout, National Cancer Institute, Bethesda Md.), were maintained by in vivo serial passages in syngeneic Balb/c mice, as described.21

Scanning electron microscopy. Cells were seeded at a density of 105 on 12-mm-round glass coverslips or 10 mm-round polycarbonate membrane filters (0.4 or 3μm pore size) in 12-well transwells (Costar, Cambridge, Mass.), and grown for 72h in the presence or absence of CsA. To assess the ability of TGF-β specific antibody to inhibit CsA-mediated effects, A-549 cells were incubated in the presence of both CsA and monoclonal antibody (1D11) (Genzyme, Boston Mass.) that neutralized TGF-β1, -β2 and -β3. Cells were fixed with PBS, pH 7.4, containing 2.0% glutaraldehyde, and processed as previously described (Ref 17). Samples were examined using a JEOL 25SIII electron microscope.

Quantification of TGF-β. TGFβ was quantified using a sandwich ELISA method as previously described (Reference 11). In brief, each well of multiwell ELISA plates was coated with anti-TGF-β1 mAb (1 μg/ml). The plates were incubated for 2 h at 37° C. after addition of various amounts of TGF-β1 in PBS or conditioned medium. After washing with PBS containing 0.2% Tween-20 (PBST), rabbit antiserum against TGF-β was added to each well. The plates were incubated at 37° C. for 1 h, and the wells were washed with PBST, and then 100 μl of goat anti-rabbit IgG-alkaline phosphatase conjugates was added. O.D. at 430 nm was measured using an ELISA reader. A-549 cells were cultured in serum-free medium to exclude contamination of cell-free supemates by serum-derived TGF-β.

Cell proliferation assay. For assaying anchorage-dependent growth, A-549 cells were grown at a density of 2×10⁴ cells/well in 12-well plates in the presence or absence of CsA. At the end of 96 h treatment, each well received 2 μCi of methyl-3H-thymidine, and cells were incubated for an additional 4 h. Cells were washed twice with ice-cold PBS, and fixed with methanol for 60 min. After washing, the fixed cells were lysed with 0.2M NaOH and treated with cold 10% TCA for 15-20 min on ice. The radioactivity, recovered as cold TCA-insoluble precipitates, was used for measuring relative cell proliferation by comparing the radioactivity between control and experiment. For an anchorage-independent cell growth, cells spread well on agarose gel were counted using a phase-contrast microscope.

In vivo tumor growth. The effect of anti-TGF-β mAb and the control IgG1 mAb on CsA-induced increase in the number of pulmonary metastases was determined by intraperitoneal administration of 200 μg of mAb, on a daily basis starting from day -1 to day 19 post tumor inoculation.

### Example 1

Cyclosporine induces A-549 cells to acquire an invasive phenotype and anti-TGF-β antibodies block this effect.

A non-transformed human pulmonary adenocarcinoma (A-549) cell line (ref. 8) that is not invasive in vitro was used as the indicator cell to test the hypothesis that CsA can induce an invasive phenotype. A-549 cells express functional receptors for TGF-β, and their growth and functions are regulated by TGF-β (refs. 7 and 8). The experiments with A-549 cells were established ex vivo to avoid any confounding effects of CsA-associated inhibition of in vivo immune surveillance mechanism(s).

Striking morphological changes were observed following CsA treatment of A-549 cells. Scanning electron microscopic examination revealed that untreated A-549 cells grown on glass cover slips for 72 hrs display a cuboidal epithelial and non-invasive phenotype. In contrast, A-549 cells conditioned with 1 μgml⁻¹ CsA exhibit phenotypic alterations that are characteristic of invasive cells: marked membrane ruffling and formation of numerous pseudopodia.

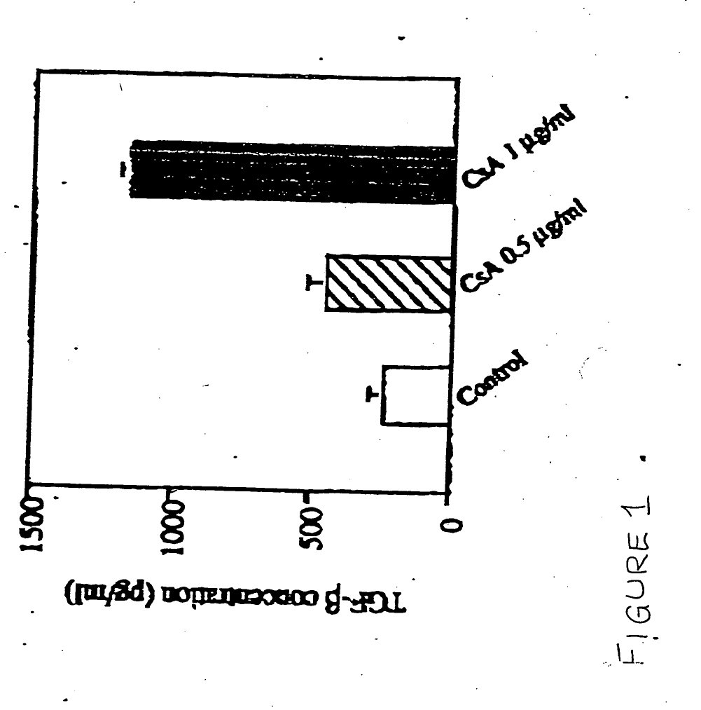

Additional data support the hypothesis that CsA-induced acquisition of an invasive phenotype is due to TGF-β protein. First, incubation of A-549 cells for 72 hrs in the absence or presence of CsA at 0.5 or 1.0 μgml⁻¹ demonstrated that CsA, in a concentration-dependent manner, stimulated TGF-β protein secretion by A-549 cells as measured by ELISA (FIG. 1). Second, anti-TGF-β mAb (30 μgml⁻¹1D11.16 IgG₁: see ref. 9), in contrast to control IgG₁ mAb, prevented CsA-induced morphological alterations. Third, addition of 2ngm⁻¹ recombinant TGF-β₁ protein induced morphological alterations in A-549 cells that were similar to those elicited by CsA, i.e. membrane ruffling and acquisition of exploratory pseudopodia. The finding that CsA stimulates TGFβ₁ production in A-549 cells extends earlier observations that CsA induces T cells, (Reference 6) CCL-64 mink lung epithelial cells (Reference 7) and renal cells (Reference 10) to hyperexpress TGF-β₁. The phenotypic changes elicited by CsA were reversible; incubation of CsA-treated A-549 cells in CsA-free culture medium for 48 h resulted in the reversal of the invasive phenotype and a return to the original morphology.

### Example 2

Cyclosporine stimulates the motility of A-549 cells and anti-TGF-β antibodies block this effect.

Cells capable of locomotion and invasiveness display exploratory pseudopodia. (References 11 and 12). Since CsA induced numerous long pseudopodia in A-549 cells, we investigated whether CsA-exposed cells acquired motility. To explore acquisition of cell locomotion, A-549 cells were grown of 72 hrs on 10 mm round polycarbonate membrane filters with three different pore sizes (0.4, 3, and 8 μm) in the presence or absence of 1 μgml⁻¹ CsA. The filters were removed from the culture dishes and the bottom surfaces of the membrane filters were examined by scanning electron microscopy. Results from this study showed that many CsA-induced pseudopodia protrude through 0.4 μm pores onto the bottom surface, whereas only few pseudopodia are seen to protrude in the control. When cells were grown on 3 μm pore filters, many CsA-treated A-549 cells, and not the untreated cells, transmigrated through the pores onto the bottom surface of the membrane filter, whereas only a few of the untreated cells migrated through the pores.

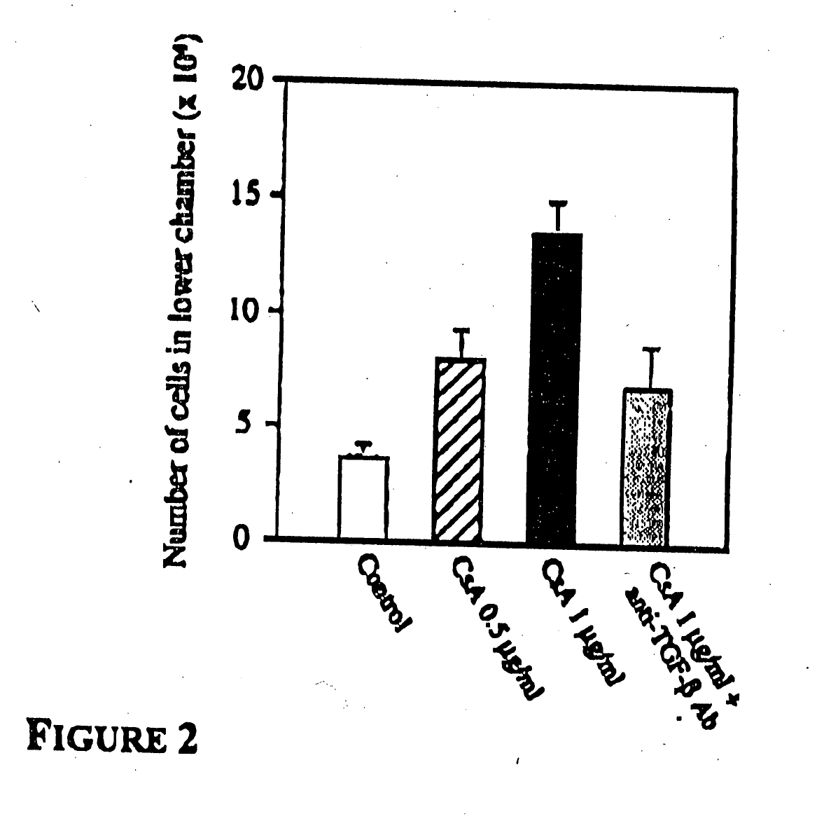

To quantify cell motility resulting from CsA-conditioning of A-549 cells, we performed a migration assay using 8 μm pore filters. A-549 cells were placed in the upper chamber of 12-well transwells (8 μm pore) at a density of 10⁵ cells, and were incubated alone or with 0.5 or 1.0 μgml⁻¹ cyclosporine or with 1.0 μgml⁻¹ cyclosporine plus 30 μgml⁻¹ anti-TGF-β antibodies (Ab). The cells that migrated into the lower chamber through the 8 μm pores of the polycarbonate membrane filters were counted after te cells had been dissociated by trypsinization. The results (mean ±s.d.) are of three experiments carried out with duplicate samples. This study demonstrated that the number of A-549 cells that migrated increased in proportion to the concentration of CsA used to treat the cells. Moreover, the increased cell motility was suppressed by the addition of 30 μgml⁻¹ anti-TGF-β mAb (FIG. 2). CsA-associated increased transmigration was not blocked by the control IgG₁ mAb. Thus, CsA-induced alterations in morphology as well as cell-motility were dependent upon CsA-induced TGF-β production.

### Example 3

Anchorage independent growth of cyclosporine conditioned A-549 cells.

Anchorage-independent growth is considered a correlate of invasive tumor growth in vivo. To assess whether CsA treatment results in anchorage-independent growth, the following study was performed: First, A-549 cells were plated on soft agarose gels and grown for 96 hrs. Culture medium (3ml) containing 10⁴ cells, in the presence or absence of 1 μgml⁻¹ CsA, was loaded onto 5ml of a 0.3% agarose layer containing MEM-10% FBS in 60mm dishes. After 96 hrs incubation at 37° C., cells grown on the surface of te agarose layer were examined with a phase-contrast microscope, revealing that untreated A-549 cells retained their spherical shape and remained suspended in the culture medium, whereas CsA-treated cells spread and proliferated well on the soft gel. Since it was difficult to determine by phase-contrast microscopy whether the pseudopodia extended along the surface of agarose or penetrated deeper into the agarose layer, we made vertical thin sections of the soft gels, and examined them by scanning electron microscopy to obtain side views. Twenty slices were made from each agarose plate. This strategy demonstrated that many grown-up pseudopodia of the CsA-treated cells penetrated the agarose gel layer and extended vertically into the gel plate. Also, CsA-treated A-549 cells appeared to be supported by the extensively invaded pseudopodia, in contrast to the lack of pseudopodial extensions in the control A-549 cells. Pseudopodia of CsA treated A-549 cells protruded deeply into the soft gel under anchorage-independent growth conditions.

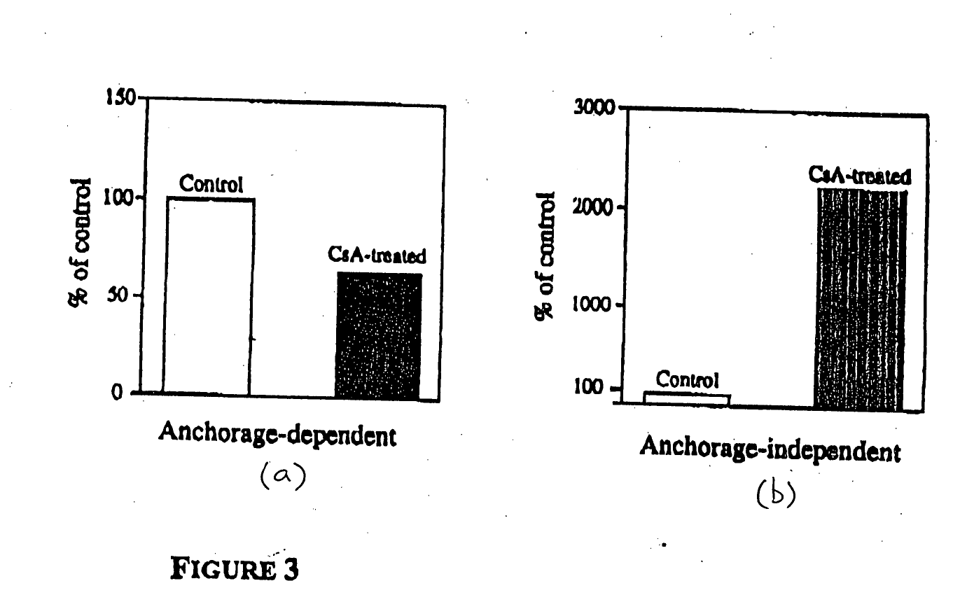

The effect of CsA on A-549 cell growth was contingent upon whether the culture conditions were anchorage-dependent or -independent. CsA inhibited proliferation of A-549 cells under anchorage-dependent conditions, whereas it stimulated proliferation under anchorage-independent conditions. (FIGS. 3a and 3b).

Further studies were performed to determine whether CsA induces morphological and functional alterations in other cell types as well. The effects of CsA on three different cell lines, murine renal cell adenocarcinoma (Renca) cells, NMuMG (mouse mammary gland epithelial) cells and CCL-64 (mink lung epithelial) cells were thus investigated. Our experiments showed that CsA treatment resulted in phenotypic alterations in these epithelial cells as well, resulting in acquisition of an invasive phenotype.

### EXAMPLE 4

Cyclosporine induces renal cancer cells to acquire an invasive phenotype and promotes tumor growth in vivo.

The following experiments were performed to determine whether CsA would enhance invasive and metastatic growth of tumor cells in vivo. In addition to Renca cells, (Reference 13) two different tumor cell lines, one of murine origin (Lewis lung carcinoma cells, reference 14) and the other of human origin (human bladder transitional carcinoma cells, reference 15), were utilized as the tumor inoculum. SCID-beige mice (mice homozygous for both SCID and beige mutations, reference 16) which are deficient in T cell, B cell and NK cells were used as the tumor-bearing host. The use of SCID-beige mice minimized the confounding variable of CsA-induced depression of host immune effector mechanisms contributing to tumor progression.

Specifically, murine renal cell adenocarinoma (Renca) cells, murine Lewis lung carcinoma (LLC) cells, or human bladder cancer (T24) cells (1×10⁵ cells each in Hanks balanced salt solution) were injected via the tail vein into 6-wk old male SCID-beige mice. CsA (20 mg/kg in 0.2 ml olive oil) was administered every other day starting from day-1, to the day of sacrifice. On day 16 (LLC), day 19 (Renca) or 23 (T24) post tumor inoculation, mice were sacrificed and the number of pulmonary metastases was determined (Reference 18) following endotracheal insufflation of lungs with 15% India ink solution and bleaching the harvested lungs in Fekete's solution.

CsA increased the number of murine renal cell cancer metastases in SCID-beige mice. Data from four separate experiments demonstrating that CsA treatment resulted in a significant increase in the number of renal cell cancer pulmonary metastases from 241±22 (mean ±SEM, n=21) in the control SCID-beige mice to 338±26 (n=18) in the CsA-treated mice (p=0.007, t test). The data are summarized in Table 1. It is also shown in Table 1 that the number of pulmonary metastases resulting from murine Lewis lung carcinoma cells (LCC) increased from 11±2 (n=9 mice) in the control mice to 28±4 (n=8 mice) with CsA treatment (p=0.003). The number of pulmonary metastases resulting from human bladder transitional cancer cells increased from 63±18 (n=9 mice) in the control mice to 138±21 (n=9 mice) in CsA-treated mice (p=0.01) (Table 1).

TABLE 1
CsA increases pulmonary metastases in SCID-beige mice.

Number of pulmonary metastases
(mean ± SEM)

| | | | | |
|---|---|---|---|---|
| Tumor Inoculum | − | CsA | + | p* |

| | | | |
|---|---|---|---|
| Murine Renca | 241 ± 22 | 338 ± 26 | 0.007 |
| | (n = 21) | (n = 18) | |
| Murine LLC | 11 ± 2 | 28 ± 4 | 0.003 |
| | (n = 9) | (n = 8) | |
| Human Bladder Cancer (T24) | 63 ± 18 | 138 ± 21 | 0.01 |
| | (n = 9) | (n = 9) | |

( ) = number of mice in each group. p value derived with t test.

### Example 5

Efects of anti-TGF-β antibodies on the cyclosporine-induced increase in metastases.

The effect of anti-TGF-β mAb (1D11.16 IgG₁ mAb, reference 9) on the CsA-induced increase in the number of metastases was also investigated. Anti-TGF-β mAb but not control IgG₁ mAb, prevented the CsA-induced increase in the number of metastases. The number of pulmonary metastases was 350±22 (mean ±SEM, n=12) in the control mice; 441±20 (n=10) in CsA-treated mice; 284±34 (n=8) in the CsA plus anti-TGF-β mAb treated mice; and 490±56 (n=4) in CsA plus control IgG₁ mAb treated mice (p=0.0005, one-way ANOVA). The reduction in the number of metastases found following administration of anti-TGF-β mAb to CsA-treated mice was significant at p<0.01 by ANOVA (Bonferoni p value). In contrast, there was no significant difference between the number of metastases found in CsA-treated mice and that found in mice treated with CsA plus control IgG₁ (p>0.05).

### References

1. Penn I. Cancers following cyclosporine therapy. Transplantation 43, 32-5 (1987)

2. Yokoyama I, Carr B, Saitsu H, Iwatsuki S, Starzl TE. Accelerated growth rates of recurrent hepatocellular carcinoma after liver transplantation. Cancer 68, 2095-2100 (1991)

3. London N J, Fannerry S M, Will E J, Davison A M, Lodge J P A. Risk of neoplasia in renal transplant patients. Lancet 346, 403-406 (1995)

4. Suthanthiran M, Strom T B. Renal transplantation. N Engl J Med 331, 365-376 (1994)

5. Kahan B D. Cyclosporine N Engl J Med 321, 1725-1738 (1989)

6. Li B, Sehajpal P K, Khanna A, Vlassara H, Cerami A, Suthanthiran M. Differential regulation of transforming growth factor β and interleukin 2 genes in human T cells: Demonstration by usage of novel competitor DNA constructs in quantitative polymerase chain reaction. J Exp Med 174, 1259-1262 (1991)

7. Khanna A, Li B, Stenzel K H, Suthanthiran M. Regulation of new DNA synthesis in mammalian cells by cyclosporine. Transplantation 57, 577-582 (1994)

8. Mooradian D L, McCarthy J B, Komanduri K V, Furcht L T. Effects of transforming growth factor- β1 on human pulmonary adenocarcinoma cell adhesion, motility, and invasion in vitro. J Natl Cancer Inst 84, 523-527 (1992)

9. Dasch J R, Pace D R, Waigell W, Inenaga D, Ellingsworth L. Monoclonal antibodies recognizing transforming growth factor-β2. Bioactivity neutralization and transforming growth factor-β2 affinity purification. J lmmunol 142, 1536-1541 (1989)

10. Wolf G, Thaiss F, Stahl R A. Cyclosporine stimulates expression of transforming growth factor-β in renal cells: Possible mechanism of cyclosporine's anti-proliferative effects. Transplantation 60, 237-241 (1995) p0 11. Guirguis R, Margulies I, Taraboletti G, Schiffinann E, Liotta L. Cytokine-induced pseudopodial protrusion is coupled to tumor cell migration. Nature 329, 261-263 (1987).

12. Couchman J R, Lenn M, Rees D A. Coupling of cytoskeletal functions for fibroblast locomotion. Eur J Cell Biol 36, 182-194 (1985)

13. Murphy G P, Hrushesky W J. A murine renal cell carcinoma. J Natl Cancer Inst 50, 1013-1025 (1973)

14. Bertram J S, Janik P. Establishment of a cloned line of Lewis Lung Carcinoma cells adapted to cell culture. Cancer Lett 11, 63-73 (1980)

15. Bubenik J, Baresova M, Viklicky V, Jakoubkova J, Sainerova H, Donner J. Established cell line of urinary bladder carcinoma (T24) containing tumour-specific antigen. Int J Cancer 11, 765-773 (1973)

16. Mosier D E, Stell K L, Gulizia R J, Torbett B E, Gilmore G L. Homozygous scid/scid:beigefbeige mice have low levels of spontaneous or neonatal T cell-induced B cell generation. J Exp Med 177, 191-194 (1993)

17. Cohen-Gould L, Robinson T F, Factor S M. Intrinsic connective tissue abnormalities in the heart muscle of cardiomyopathy Syrian hamsters. Am J Pathol 127, 327-334 (1987)

18. Asano T, Khanna A, Lagman M, Li B, Suthanthiran M. Immunostimulatory therapy with anti-CD3 monoclonal antibodies and recombinant interleukin-2: Heightened in vivo expression of mRNA encoding cytotoxic attack molecules and immunoregulatory cytokines and regression of murine renal cell carcinoma. J Urol 157, 2396-2401 (1997)

1. A method for reducing formation or progression of neoplasms associated with immunosuppressive therapy in a mammal, the method comprising treating the mammal with an effective amount of a TGF-β antagonist.

2. The method according to claim 1, wherein the TGF-β antagonist inhibits the activity of TGF-β1.

3. The method according to claim 1, wherein the TGF-β antagonist inhibits the activity of TGF-β2.

4. The method according to claim 1, wherein the TGF-β antagonist inhibits the activity of TGF-β3.

5. The method according to claim 1, wherein the TGF-β antagonist inhibits the activity of more than one isoform of TGF-β.

6. The method according to claim 1, wherein the TGF-β antagonist comprises a protein or polypeptide.

7. The method according to claim 1, wherein the TGF-β antagonist comprises an antibody directed against TGF-β.

8. The method according to claim 1, wherein the TGF-β antagonist comprises a TGF-β receptor or fragment thereof.

9. The method according to claim 1, wherein the TGF-β antagonist inhibits the activity of a TGF-β receptor.

10. The method according to claim 9, wherein the TGF-β receptor is TGF-β receptor type 1.

11. The method according to claim 9, wherein the TGF-β receptor is TGF-β receptor type 2.

12. The method according to claim 9, wherein the TGF-β receptor is TGF-β receptor type 3.

13. The method according to claim 9, wherein the TGF-β antagonist is an antibody or an antibody fragment specific for the TGF-β receptor.

14. The method according to claim 1, wherein the mammal is a human.

15. The method according to claim 1, wherein the immunosuppressive therapy comprises treatment with cyclosporine.

16. The method according to claim 1, wherein the immunosuppressive therapy comprises treatment with FK506.

17. The method according to claim 1, wherein the TGF-β antagonist is administered prior to immunosuppressive therapy.

18. The method according to claim 1, wherein TGF-β antagonist is administered during immunosuppressive therapy.

19. The method according to claim 1, wherein the TGF-β antagonist is administered after immunosuppressive therapy.

20. The method according to claim 1, wherein the TGF-β antagonist administration overlaps the period of immunosuppressive therapy.

21-35. (Cancelled).
